(19) United States
(12) Patent Application Publication
(10) Pub. No.: US 2016/0271415 A1
(43) Pub. Date: Sep. 22, 2016

(54) TEETH WHITENING APPARATUS

(71) Applicant: BS and Co. Ltd., Seoul (KR)

(72) Inventor: Sang Ki Min, Gyeonggi-do (KR)

(21) Appl. No.: 14/785,516

(22) PCT Filed: Sep. 11, 2014
(86) PCT No.: PCT/KR2014/008484

§ 371 Date: Jun. 3, 2016

(30) Foreign Application Priority Data
Aug. 29, 2014 (KR) 10-2014-0114053

(51) Int. Cl. A61N 5/06; A61C 19/06

(52) U.S. Cl. CPC A61N 5/0603; A61N 5/062; A61C 19/066; A61N 2005/0606; A61N 2005/0632; A61N 2005/0643; A61N 2005/063

(57) ABSTRACT

Disclosed is herein a teeth whitening apparatus. The teeth whitening apparatus according to the present invention includes a mouth gap inserted into a user's oral cavity so as to be in close contact with his or her teeth, and a main body connected to the mouth gap by means of a cable and configured to supply power to the mouth gap, the main body having a switch configured to control an ON/OFF operation, wherein the mouth gap includes: a body formed in a flat rectangular parallelepiped shape which is curved in its entirety, the body having an accommodating space formed therein and an opening formed at one side thereof; a light-emitting unit mounted in the accommodating space of the body and including a PCB embedded with a plurality of light-emitting devices; and an oral cavity wearing unit including a coupling member detachably coupled to the outer periphery of the opening of the body and having a vent aperture formed at the center thereof and an insertion member formed at one side of the coupling member so as to be inserted into the user's mouth and brought into close contact with his or her teeth, and wherein the insertion member comprises a pair of opposed holding protrusions protrudingly formed inwardly from the inner surface thereof so as to be held between his or her upper and lower teeth, the insertion member having one or more elongated vent holes formed thereon in such a manner as to be perforated in a predetermined length to have air permeability.

### CROSS-REFERENCE TO RELATED APPLICATION

This Application is a Section 371 National Stage Application of International Application No. PCT/KR2014/008484, filed on Sep. 11, 2014, the contents of which is hereby incorporated by reference in its entirety.

### TECHNICAL FIELD

The present invention relates to a teeth whitening apparatus, and more particularly, to a teeth whitening apparatus in which a user's mouth is opened using a mouth gap made of a silicon material after applying a teeth whitening agent on his or her teeth to remove an environment capable of blocking infiltration of light, and light emitted from a light-emitting device is irradiated onto the teeth to exhibit a teeth whitening function by a photocatalyst, and particularly oxygen is smoothly induced to be supplied to a mouth gap so that the whitening function can be promoted by the photocatalyst.

### BACKGROUND ART

In recent years, the colour of teeth is recognized as a part of cosmetic applications along with an increase in the interest on plastic surgery and beauty.

Human teeth are likely to be discolored to quite or blackish yellow due to various causes such as drug, inherent factor, cigarette, food color, and the like.

Currently, many people have an interest on making their lives beautiful along with an improvement of living standards. In particular, teeth whitening is drawing an attention as an aesthetic treatment among young women.

Teeth whitening is an aesthetic dental procedure by which the teeth colour becomes a shade lighter than the existing one, which gives a clean image to a person subjected to as teeth whitening treatment as well as a counterparty communicating with him or her, and thus many people have an increasing interest on the teeth whitening.

The teeth whitening treatment is basically performed in such a manner that a user brushes his or her teeth with a toothpaste containing a teeth whitening agent to obtain a teeth whitening effect. However, since the teeth whitening agent is contained in the toothpaste in a quite small amount, a coloring component deposited on the surfaces of the teeth could not be removed effectively.

Thus, for the purpose of a more effective whitening treatment, a method is employed in which a patient undergoes a professional dental procedure using a whitening agent pad or a laser in a dental clinic.

Such a method entails a problem in that the whitening effect is excellent but the patient suffers from an inconvenience of having to go to the dental clinic regularly for a long period of time and a high treatment cost is consumed.

In an attempt to solve the above problem, there has been proposed a whitening patch as an eclectic alternative which is convenient to treat and has an excellent effect.

The whitening patch is a product in which an ointment agent containing a whitening component based on low-concentration carboxide or peroxide is applied on the surface of the patch. The whitening patch enables the sufficient and long-term use of the whitening component to exhibit an excellent whitening effect as well as the preparation for dental treatment is terminated only by attachment of the whitening patch on the front and rear surfaces of the teeth according to use instructions and thus anyone can use the whitening patch easily.

Such a whitening patch, however, involves a problem in that since a sticking portion attached to the teeth is water soluble, the patch is shifted from an original attachment position due to movement of muscles around a mouth during the long-term use thereof and an ointment portion thereof stimulates or damages a tongue or a mucous membrane of an oral cavity.

In order to supplement the above problem, a light emission type teeth whitening apparatus has been currently developed.

The light emission type teeth whitening apparatus is configured such that a whitening agent containing a photoactivator is applied on a user's teeth, and an active light beam is irradiated to the whitening agent applied on the teeth in a structure in which oxygen is supplied smoothly so that a whitening treatment can be promoted.

Meanwhile, as one example of the prior art, there has been disclosed Korean Patent Registration No. 10-0773379 entitled “Apparatus for Whitening Teeth”.

The teeth whitening apparatus according to the prior art includes: a casing having an opening formed at one side thereof; a light-emitting unit including a light-emitting device mounted in the casing and a light-scattering member mounted at the opening of the casing and configured to scatter light emitted from the light-emitting device; and a mouth gap configured to transmit the scattered light to a user's teeth.

By the way, it is required that a photocatalyst should be supplied the teeth to promote a whitening property by light irradiation and oxygen should be supplied smoothly to facilitate a catalytic reaction.

However, the conventional teeth whitening apparatus encounters a problem in that since the mouth gap has a closed structure, oxygen is not smoothly supplied, thereby degrading a whitening function.

### DISCLOSURE

### Technical Problem

Accordingly, the present invention has been made to solve the above-mentioned problems, and an objective of the present invention is to provide a teeth whitening apparatus in which a user's mouth is opened using a mouth gap made of a silicon material after directly applying a teeth whitening agent on his or her teeth to remove an environment capable of blocking infiltration of light, and light emitted from a light-emitting device is irradiated onto the teeth to exhibit a teeth whitening function by a photocatalyst, and in which particularly a mouth gap is more easily held between his or her teeth, the user can breathe more easily, and the luminous intensity of light emitted from the light-emitting device is focused on a specific point, thereby improving the teeth whitening function.

### Technical Solution

To accomplish the above object, in accordance with the present invention, there is provided a teeth whitening apparatus including a mouth gap inserted into a user's oral cavity so as to be in close contact with his or her teeth, and a main body connected to the mouth gap by means of a cable and configured to supply power to the mouth gap, the main body having a switch configured to control an ON/OFF operation, wherein the mouth gap includes: a body formed in a flat rectangular parallelepiped shape which is curved in its entirety, the body having an accommodating space formed therein and an opening formed at one side thereof; a light-emitting unit mounted in the accommodating space of the body and including a PCB embedded with a plurality of light-emitting devices; and an oral cavity wearing unit including a coupling member detachably coupled to the outer periphery of the opening of the body and having a vent aperture formed at the center thereof and an insertion member formed at one side of the coupling member so as to be inserted into the user's mouth and brought into close contact with his or her teeth, and wherein the insertion member includes a pair of opposed holding protrusions protrudingly formed inwardly from the inner surface thereof so as to be held between his or her upper and lower teeth, the insertion member having one or more elongated vent holes formed thereon in such a manner as to be perforated to have air permeability.

In accordance with a preferred embodiment of the present invention, the coupling member includes a plurality of engagement protrusions formed on the outer periphery thereof so as to be engaged with a plurality of engagement holes formed on an engagement member of the body.

The light-emitting unit is disposed so as to be overlapped with the PCB and comprises a light guide plate having a plurality of diffusion lenses convexly formed on one surface thereof to respectively correspond to the light-emitting devices, the light guide plate being formed in an accurately curved shape and having a light transmission property. In addition, each of the diffusion lenses 152 has an apical portion 154 formed in a flat shape so that light can be propagated in a straight line.

The light guide plate has a plurality of abutting plates formed on the other surface thereof in such a manner as to be partitioned by a plurality of slits.

The plurality of slits form a honeycomb structure and each of the plurality of abutting plates is formed in a hexagonal shape.

The main body is formed in a generally rectangular parallelepiped shape having a predetermined area, the main body including a recessed portion formed concavely on the top surface thereof so allow the body to be seated therein, a light-transmitting plate formed on the recessed portion, and a UV light emitting body mounted therein to allow light emitted therefrom to be transmitted through the light-transmitting plate.

The main body includes a USB charging terminal formed at one side thereof, a connection jack formed at one side thereof to allow a connection terminal formed at an end of the cable to be inserted thereto, and an indicator lamp formed at one side thereof so as to indicate a charge level.

### Advantageous Effect

In accordance with an embodiment of the present invention, light emitted from a light-emitting device is irradiated onto a user's teeth to exhibit a teeth whitening function, oxygen is induced to be smoothly supplied to the teeth on which a whitening agent is applied so that the teeth whitening function can be promoted by a photocatalyst and an oral cavity wearing unit can be detachably mounted to a body to replace an old oral cavity wearing unit with new one. Thus, the teeth whitening apparatus according to the present invention can be used by several people using one mouth gap, thereby obtaining an economic effect.

### BRIEF DESCRIPTION OF THE DRAWINGS

The above objects, other features and advantages of the present invention will become more apparent by describing the preferred embodiments thereof with reference to the accompanying drawings, in which:

FIG. 1 is a perspective view showing a teeth whitening apparatus in accordance with an embodiment of the present invention.

FIG. 2 is a front view showing a mouth gap of a teeth whitening apparatus in accordance with an embodiment of the present invention.

FIGS. 3 to 5 are exploded perspective views showing a mouth gap of a teeth whitening apparatus in accordance with an embodiment of the present invention.

FIG. 6 is a perspective view showing a main body of a teeth whitening apparatus in accordance with an embodiment of the present invention.

FIG. 7 is a perspective view showing the coupling state of a teeth whitening apparatus in accordance with an embodiment of the present invention.

*Explanation on reference numerals
of main elements in the drawings *

| | |
|---|---|
| 100: body | 110: front member |
| 120: PCB | 122: light-emitting device |
| 130: engagement member | 150: light guide plate |
| 152: diffusion lens | 156: abutting plate |
| 200: light-emitting unit | 300: oral cavity wearing unit |
| 310: coupling member | 330: protrusions |

### PREFERRED EMBODIMENTS OF THE INVENTION

Now, preferred embodiments of the present invention will be described in detail with reference to the accompanying drawings. The matters defined in the description, such as the detailed construction and elements, are nothing but specific details provided to assist those of ordinary skill in the art in a comprehensive understanding of the invention, and the present invention is not limited to the embodiments disclosed hereinafter.

In the drawings, for the sake of a clearer understanding of the present invention, sizes or shapes of elements illustrated in the drawings may be exaggerated for clarity and convenience of explanation. Also, the following terms particularly defined in consideration of the configuration and function of the present invention may vary according to an intention of a user or an operator or according to custom. Therefore, definition of such terms should be made based on overall contents of the specification.

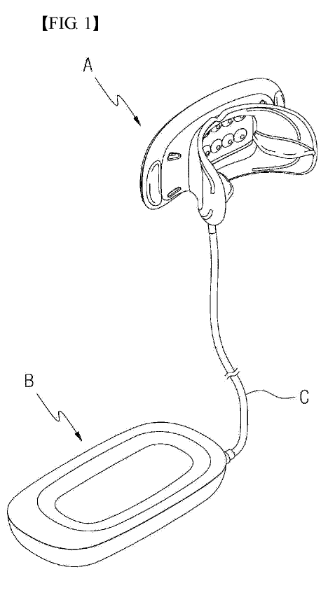

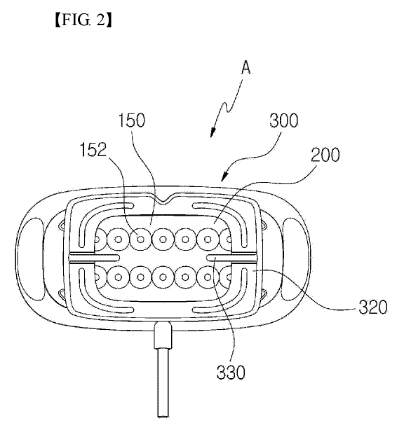

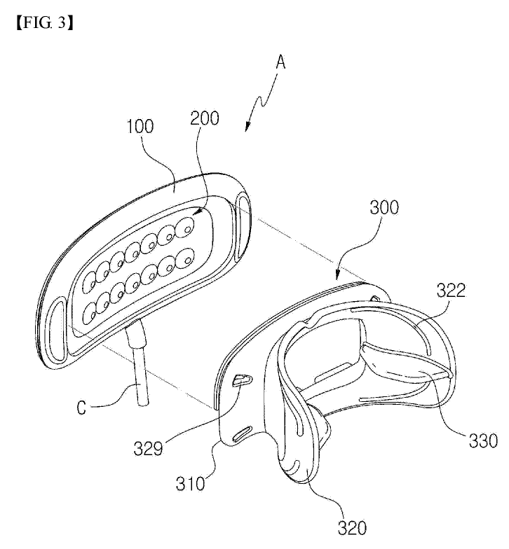

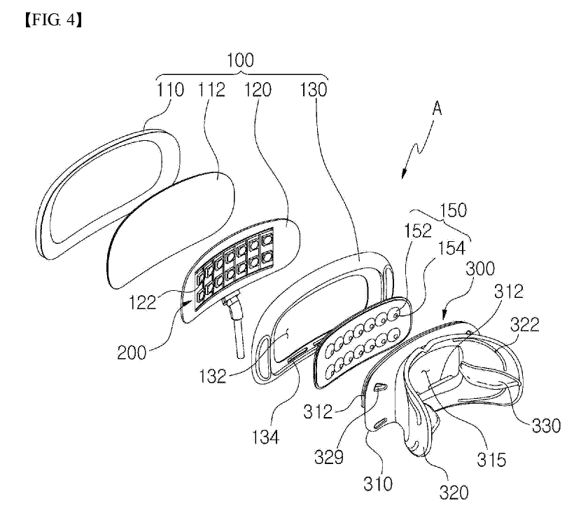

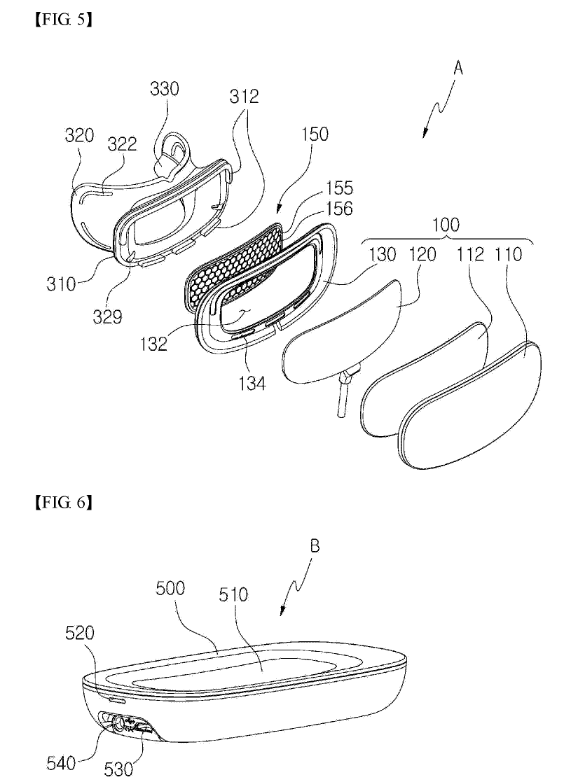

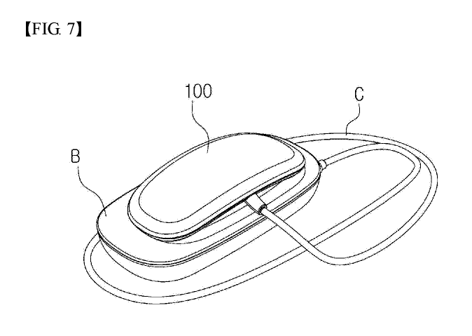

In the accompanying drawings, FIG. 1 is a perspective view showing a teeth whitening apparatus in accordance with an embodiment of the present invention, FIG. 2 is a front view showing a mouth gap of a teeth whitening apparatus in accordance with an embodiment of the present invention, FIGS. 3 to 5 are exploded perspective views showing a mouth gap of a teeth whitening apparatus in accordance with an embodiment of the present invention, FIG. 6 is a perspective view showing a main body of a teeth whitening apparatus in accordance with an embodiment of the present invention, and FIG. 7 is a perspective view showing the coupling state of a teeth whitening apparatus in accordance with an embodiment of the present invention.

The present invention is directed to a light emission type teeth whitening apparatus. In other words, the light emission type teeth whitening apparatus is configured such that a whitening agent containing a photoactivator is applied on a user's teeth, and an active light beam is irradiated to the whitening agent applied on the teeth so that a whitening treatment can be promoted by activating the whitening agent through the photoactivator.

As shown in FIGS. 1 to 7, the teeth whitening apparatus according to the present invention includes a mouth gap A inserted into a user's oral cavity so as to be in close contact with his or her teeth, and a main body B connected to the mouth gap A by means of a cable C and configured to supply power to the mouth gap A, the main body B having a switch configured to control an ON/OFF operation.

The mouth gap A includes:

a body 100 formed in a flat rectangular parallelepiped shape which is curved in its entirety, the body having an accommodating space formed therein and an opening 132 formed at one side thereof;

a light-emitting unit 200 mounted in the accommodating space of the body 100 and including a PCB 120 embedded with a plurality of light-emitting devices 122;

an oral cavity wearing unit 300 including a coupling member 310 detachably coupled to the outer periphery of the opening 132 of the body and having a vent aperture 315 formed at the center thereof and an insertion member 320 formed at one side of the coupling member 310 so as to be inserted into the user's mouth and brought into close contact with his or her teeth.

The body 100 includes a front member 110 that is formed in a generally rectangular arcuate shape, a shield plate 112 that is inserted into the front member 110 so as to be in contact with the front member 110 and shields transmission of light, and an engagement member 130 that is coupled to the front member 110 and has an opening 132 formed at the center thereof and a plurality of engagement holes 134 formed at a lower portion thereof.

Thus, the body 100 has a generally rectangular opening 132 formed at one side thereof to allow that the light-emitting devices 122 of the light-emitting unit 200 to be exposed to the outside therethrough so that light emitted from the light-emitting devices 122 can be transmitted to the oral cavity wearing unit 300 through the opening 132.

The light-emitting unit 200 is disposed so as to be overlapped with the PCB 120 and includes a light guide plate 150 having a plurality of diffusion lenses 152 formed thereon to respectively correspond to the light-emitting devices 122.

Each of the light-emitting devices 122 preferably is an LED that has various colors and luminous intensities.

The LED emits light of 20% and heat of 80% in terms of its properties, and requires a heat dissipation structure in view of the fact that the mouth gap A is inserted into the user's oral cavity.

The light guide plate 150 is formed in an accurately curved shape and is made of a synthetic resin material having a light transmission property to provide a light scattering and diffusing function.

The light guide plate 150 has a plurality of diffusion lenses 152 convexly formed on one surface thereof, and each of the diffusion lenses 152 has an apical portion 154 formed in a flat shape so that light can be propagated in a straight line.

The diffusion lenses 152 are formed to be protruded convexly in a hemi-spherical shape, and are arranged to respectively correspond to the light-emitting devices 122 in a one-to-one relationship.

In addition, the light guide plate 150 is made of a translucent material such as a light diffusion ABS resin, and has a plurality of abutting plates 156 formed on the other surface thereof in such a manner as to be partitioned by a plurality of slits 155.

The plurality of slits 155 is arranged continuously in a hexagonal shape to form a honeycomb structure.

Thus, each of the plurality of abutting plates 156 defined by the plurality of slits 155 is formed in a hexagonal shape.

The abutting plates 156 formed on the other surface of the light guide plate 150 are arranged to respectively correspond to the light-emitting devices 122 in a one-to-one relationship, and simultaneously are arranged to respectively correspond to the diffusion lenses 152 in a one-to-one relationship.

Thus, light emitted from one light-emitting device 122 stays in the corresponding abutting plates 156 and then is diffused through the corresponding diffusion lenses 152.

In addition, high intensity light is irradiated focusingly through the apical portions 154 of the diffusion lenses so that the light is directly irradiated to the user's teeth.

Further, a part of light is diffusingly delivered to the diffusion lenses 152 and the light guide plates 150 in their entirety so that the light is irradiated to the teeth.

Light is converted to have various intensities by the diffusion lenses 152 and the abutting plates 156 so that a direct irradiation and an indirect irradiation of light are performed, and a function of intensively and totally whitening the teeth can be exhibited while allowing the light to be reacted with the whitening agent.

In the meantime, since the insertion member 320 of the oral cavity wearing unit 300 is brought into close contact with the user's teeth and gums, it is preferably made of a soft material such as silicon that is harmless to the human body and is formed in an arcuately curved shape which encircles the entire surface of the teeth.

Furthermore, the insertion member 320 has a pair of opposed protrusions 330 protrudingly formed inwardly from the inner surface thereof so that the protrusions 330 can be held between the user's back teeth with him or her biting the protrusions.

The pair of protrusions 330 is protruded horizontally and is made of a soft synthetic resin material.

In addition, the insertion member 320 is formed in a generally rectangular actuate shape and has a plurality of elongated vent holes 322 formed at the upper and lower portions thereof in such a manner as to be perforated in a predetermined length to have air permeability.

The coupling member 310 additionally has a plurality of oxygen inflow holes 329 symmetrically formed thereon.

The coupling member 310 has a plurality of engagement protrusion 312 formed on the outer periphery thereof to allow the engagement protrusion 312 to be engaged with the engagement holes 134 of the engagement member 130 of the body 100.

The engagement protrusions 312 are formed at the upper and lower portions of a front surface of the coupling member 310 such that the engagement protrusion 312 formed at the upper portion of the coupling member 310 is formed in a hooked shape which is bent downwardly so as to be fittingly inserted into and brought into close contact with the engagement holes 134 of the engagement member 130, and the engagement protrusion 312 formed at the lower portion of the coupling member 310 is formed protrudingly in a straight shape so as to be engaged with the engagement holes 134 of the engagement member 130 in a press-fit engagement manner.

Meanwhile, the main body B is formed in a generally rectangular parallelepiped shape having a predetermined area. The main body B has a recessed portion 500 formed concavely on the top surface thereof so allow the body 100 to be seated therein, a light-transmitting plate 510 formed on the recessed portion 500, and a UV light emitting body (not shown) mounted therein to allow light emitted therefrom to be transmitted through the light- transmitting plate 510.

The main body B includes a USB charging terminal 530 formed at one side thereof, a connection jack 540 formed at one side thereof to allow a connection terminal formed at an end of the cable C to be inserted thereto, and an indicator lamp 520 formed at one side thereof so as to indicate a charge level of a battery.

The USB charging terminal 530 is configured such that a five-pin terminal block of a standardized cellular phone recharger is coupled to the terminal 530, and can employ a power supply of 110V or 220V.

In addition, the main body B includes a control device that can control the battery (not shown) and the UV light emitting body to be operated for eight minutes.

It is to be, of course, noted that the operating time of the control device can vary variously depending on the need.

In the meantime, the main body B may additionally include a magnetic attachment means so that the body is seated on the recessed portion 500 and then is fixedly attached to the recessed portion 500 by a magnetic force.

In other words, the magnetic attachment means is composed of a first magnet disposed in the recessed portion 500 and a second magnet disposed in the engagement member 130 of the body 100 to allow the first magnet and the second magnet to be magnetically attached to each other so that the body 100 can be prevented front losing and arrangement of the teeth whitening apparatus can be performed easily.

The operation of the teeth whitening apparatus according to the present invention as constructed above will be described hereinafter.

After a whitening agent is directly applied on a user's teeth using a lipstick shaped whitening agent container (not shown) having a whitening agent contained therein, the oral cavity wearing unit 300 of the mouth gap A is inserted into his or her oral cavity and is held between his or her upper and lower teeth.

Preferably, the user's lips are positioned on the upper and lower surfaces and both sidewalls of the oral cavity wearing unit 300 so that his or her saliva is prevented from being introduced into the mouth gap A.

About 20-30 minutes are sufficient for a gel-state whitening agent to be applied on the front and side surfaces of the user's teeth.

After the whitening agent is evenly and surroundingly applied on the front and side surfaces of the user's teeth, a whitening reaction against a coloring component deposited on the user's teeth begins to be carried out by a whitening component such as a photocatalyst contained in the whitening agent.

A teeth whitening composition of the whitening agent contains an active ingredient-containing composition that achromatizes a teeth coloring chromophore or reduce the coloring of the teeth, or a composition that can dissolve the teeth coloring chromophore so as to be more vulnerable to the mechanical or chemical removal.

Examples of the active ingredient suitable for this purpose include hydrogen peroxide (or any hydrogen peroxide precursor material), carbamide peroxide, sodium percarbonate, calcium peroxide, sodium perborate, potasium persulfate, peracetic acid (or other peracid), and chlorine dioxide.

Preferably, the teeth whitening composition is used simultaneously with an activation means that increases the rate and efficiency thereof.

The activation means includes a light source, a heat source, or a composition (i.e., activation composition) that is independently of a teeth whitening composition that chemically increases the rate and efficiency of the teeth whitening composition.

The activation composition may contain metal ions, a chelate complex compound of the metal ions, and a peracid precursor material (i.e., material capable of forming peracid under the existence of hydrogen peroxide such as glyceryl triacetate).

The light source is preferably positioned so as to be parallel with the occlusal plane of teeth in a state in which the oral cavity wearing unit is inserted into the user's oral cavity and the a pair of protrusions 330 is held between his or her upper and lower teeth.

Thereafter, when the switch of the main body is turned on, the light-emitting units disposed in the mouth gap are lit and light emitted from the LEDs is irradiated to the user's teeth.

Thus, the photoactivator contained in the whitening agent is activated by an optical energy of the irradiated light to promote the whitening reaction of the whitening component so that the teeth whitening treatment time can be shortened and simultaneously the teeth whitening effect can be enhanced.

There has been proposed a method of using an LED lighting for the treatment of a cutaneous wound, in which LED light having a wavelength range of about 620-680 nm is used.

In the present invention, LED light having a wavelength range of 400-500 nm that is the most suitable for the teeth whitening is irradiated to the user's teeth so that a combined sterilizing and teeth whitening function can be exhibited.

Preferably, in case of the power density of light delivered to the user's teeth, a sum of energy densities of light supplied for eight minutes ranges from 20 J/cm² to 2 W/cm². A preferable wavelength of light emitted from the light-emitting unit is set to a range of 400 nm to 505 nm.

Preferably, light emitted from the light-emitting unit is illuminated for approximately 5-8 minutes. The light source is preferably set such that it is extinguished automatically after the illumination period time.

Accordingly, the photoactivator contained in the whitening agent is activated by the optical energy of the irradiated light to promote the whitening reaction of the whitening component so that the teeth whitening treatment time can be shortened and simultaneously the teeth whitening effect can be enhanced.

Moreover, air is introduced into or discharged from the user's oral cavity in a respiration process. Likewise, when the flow of air occurs, air mostly flows via the elongated vent holes formed on the oral cavity wearing unit 300.

Air can be diffused to a wider range while passing through the elongated vent holes and the diffused air can be evenly jetted to the user's teeth. Thus, oxygen contained in air reacts with the whitening agent applied on his or her teeth to promote a photocatalytic reaction so that the whitening efficacy can be doubled.

A whitening cycle by the optical irradiation as described above is repeated about 2-3 times, and one whitening cycle is performed for about eight minutes.

An excessive amount of the whitening agent is removed from the user's teeth during a first cycle and then the whitening agent is re-filled for a second cycle. Light is illuminated for about eight minutes in the above manner. Preferably, a third cycle is performed in which the whitening agent is applied on the user's teeth and light is illuminated for about eight minutes to the teeth.

After the whitening treatment performed more than three times has been completed, the whitening agent is removed from the user's teeth using water to measure the shade of the user's teeth to confirm whether or not the teeth is whitened sufficiently,

In the case where an additional whitening treatment is desirable, the user can be subjected to the additional whitening treatment for about 1-8 weeks.

After the teeth whitening process has been completed, the user's teeth is treated with a desensitizing composition selected from the group including, but not limited to, potassium nitrate and gel fluoride.

### INDUSTRIAL APPLICABILITY

While the present invention has been described in connection with the specific embodiments illustrated in the drawings, they are merely illustrative, and the invention is not limited to these embodiments, it is to be understood that various equivalent modifications and variations of the embodiments can be made by a person having an ordinary skill in the art without departing from the spirit and scope of the present invention.

Therefore, the true technical scope of the present invention should not be defined by the above-mentioned embodiments but should be defined by the appended claims and equivalents thereof.

What is claimed is:

1. A teeth whitening apparatus comprising
a mouth gap inserted into a user's oral cavity so as to be in close contact with his or her teeth, and a main body connected to the mouth gap by means of a cable and configured to supply power to the mouth gap, the main body B having a switch configured to control an ON/OFF operation,
wherein the mouth gap comprises:
a body formed in a flat rectangular parallelepiped shape which is curved in its entirety, the body having an accommodating space formed therein and an opening formed at one side thereof;
a light-emitting unit mounted in the accommodating space of the body and including a PCB embedded with a plurality of light-emitting devices; and
an oral cavity wearing unit including a coupling member detachably coupled to the outer periphery of the opening of the body and having a vent aperture formed at the center thereof and an insertion member formed at one side of the coupling member so as to be inserted into the user's mouth and brought into close contact with his or her teeth, and
wherein the insertion member comprises a pair of opposed holding protrusions protrudingly formed inwardly from the inner surface thereof so as to be held between his or her upper and lower teeth, the insertion member having one or more elongated vent holes formed thereon in such a manner as to be perforated in a predetermined length to have air permeability.

2. The teeth whitening apparatus according to claim 1, wherein the light-emitting unit is disposed so as to be overlapped with the PCB and comprises a light guide plate having a plurality of diffusion lenses convexly formed on one surface thereof to respectively correspond to the light-emitting devices, and
the light guide plate is formed in an accurately curved shape and has a light transmission property.

3. The teeth whitening apparatus according to claim 2, wherein the light guide plate has a plurality of abutting plates formed on the other surface thereof in such a manner as to be partitioned by a plurality of slits.

4. The teeth whitening apparatus according to claim 3, wherein the plurality of slits form a honeycomb structure and each of the plurality of abutting plates is formed in a hexagonal shape.

5. The teeth whitening apparatus according to claim 3, wherein the abutting plates are arranged to respectively correspond to the light-emitting devices and the diffusion lenses in a one-to-one relationship.

6. The teeth whitening apparatus according to claim 3, wherein the main body is formed in a generally rectangular parallelepiped shape having a predetermined area, the main body comprising a recessed portion formed concavely on the top surface thereof so allow the body to be seated therein, a light-transmitting plate formed on the recessed portion, and a UV light emitting body mounted therein to allow light emitted therefrom to be transmitted through the light-transmitting plate.

7. The teeth whitening apparatus according to claim 5, wherein the main body comprises a USB charging terminal formed at one side thereof, a connection jack formed at one side thereof to allow a connection terminal formed at an end of the cable to be inserted thereto, and an indicator lamp formed at one side thereof so as to indicate a charge level.
